(19) United States
(12) Patent Application Publication
(10) Pub. No.: US 2020/0060663 A1
(43) Pub. Date: Feb. 27, 2020

(54) SURGICAL DEVICE CONFIGURED TO STRIP AND CUT TENDON

(71) Applicant: Arthrex, Inc., Naples, FL (US)

(72) Inventors: Jacob Jolly, Naples, FL (US); Joshua Michalik, Naples, FL (US); John Xerogeanes, Atlanta, GA (US); Kenneth T. Helenbolt, Naples, FL (US); Zachary Ingwer, Boca Raton, FL (US)

(21) Appl. No.: 16/106,758

(22) Filed: Aug. 21, 2018

(51) Int. Cl. A61B 17/00; A61B 17/3205

(52) U.S. Cl. CPC A61B 17/00008; A61B 17/32075; A61B 17/32053

(57) ABSTRACT

This disclosure relates to a surgical device configured to both strip and cut a tendon. The harvested tendon can be used in various orthopedic procedures, such as ACL, PCL, and UCL reconstructions.

### BACKGROUND

Tendons are commonly harvested for use in orthopedic procedures. In particular, tendons may be autografts, harvesting from a patient's hamstring tendon, quadriceps tendon, or other areas of the body. The harvested tendons may be used in ligament reconstruction surgeries, e.g., anterior cruciate ligament (ACL), posterior cruciate ligament (PCL), and ulna collateral ligament (UCL) reconstruction surgeries.

### SUMMARY

This disclosure relates to a surgical device configured to both strip and cut a tendon. A harvested tendon can be used in various orthopedic procedures, such as ACL, PCL, and UCL reconstructions.

A surgical device according to an embodiment of the present disclosure includes, inter alia, a stripping tube configured to strip a tendon, and a cutter moveable distally toward the distal end of the stripping tube to sever a portion of the tendon.

A method according to an embodiment of the present disclosure includes, inter alia, separating a portion of a tendon by moving a stripping tube of a surgical device distally relative to the tendon, and severing the separated portion of the tendon by moving a cutter of the surgical device distally relative to the stripping tube.

### BRIEF DESCRIPTION OF THE DRAWINGS

FIG. 1 is a side view of a first example surgical device.

FIG. 2 is a partial perspective view of the surgical device of FIG. 1, and in particular illustrates a cutter in an intermediate position between a resting position and a fully deployed position.

FIG. 3 is a partial side-perspective view of the surgical device of FIG. 1, and in particular illustrates the detail of a stripping tube.

FIG. 4 is a partial perspective view of the surgical device of FIG. 1, and in particular illustrates the cutter in a fully deployed position.

FIG. 5 is a partial side-perspective view of the surgical device of FIG. 1, and in particular illustrates the detail of the cutter.

FIG. 6 is a partial top-perspective view of the surgical device of FIG. 1, and in particular illustrates the detail of the stripping tube and an adjacent cutout.

FIG. 7 is a side view of the surgical device of FIG. 1 with a portion of a handle removed for ease of reference. In FIG. 7, a lock assembly is engaged.

FIG. 8 is a side view of the surgical device of FIG. 1 with a portion of a handle removed for ease of reference. In FIG. 8, the lock assembly is disengaged.

FIG. 9 is a side view of the surgical device of FIG. 1 with a portion of the handle removed for ease of reference. In FIG. 9, the stripping tube is in contact with a tendon and the cutter is in a resting position.

FIG. 10 is a side view of the surgical device of FIG. 1 with a portion of the handle removed for ease of reference. In FIG. 10, the cutter has advanced to an intermediate position distal of the resting position.

FIG. 11 is a side view of the surgical device of FIG. 1 with a portion of the handle removed for ease of reference. In FIG. 11, the cutter has advanced distally to a fully deployed position and severed a portion of the tendon.

FIG. 12 is a partial top view of a second example surgical device.

### DETAILED DESCRIPTION

This disclosure relates to a surgical device configured to both strip and cut a tendon. The harvested tendon can be used in various orthopedic procedures, such as ACL, PCL, and UCL reconstructions.

A surgical device according to an exemplary aspect of the present disclosure includes, inter alia, a stripping tube configured to strip a tendon, and a cutter moveable distally toward the distal end of the stripping tube to sever a portion of the tendon.

In a further embodiment, the stripping tube is tapered.

In a further embodiment, the distal edge of the stripping tube includes a plurality of serrations.

In a further embodiment, the stripping tube includes a window which is substantially circular in cross-section.

In a further embodiment, the stripping tube comprises a window allowing the portion of the tendon to pass therethrough.

In a further embodiment, the cutter is disposed circumferentially.

In a further embodiment, the distal end of the cutter includes a frustoconical recess.

In a further embodiment, the distal end of the cutter is tapered.

In a further embodiment, the cutter is moveable such that a tendon may be severed between a proximal edge of the stripping tube and a distal edge of the cutter.

In a further embodiment, the proximal edge of the stripping tube is tapered and the distal edge of the cutter is tapered.

In a further embodiment, the distal edge of the cutter includes at least one serration adjacent.

In a further embodiment, the at least one serration is a single serration substantially symmetrical about a centerline of the surgical device.

In a further embodiment, the stripping tube and cutter are disposed about a common axis.

In a further embodiment, the cutter is configured to rotate about the common axis as the cutter moves axially relative stripping tube.

In a further embodiment, the stripping tube is configured to rotate about the common axis as the stripping tube moves axially relative to the cutter.

In a further embodiment, the cutter includes a helical slot receiving a pin, the pin and helical slot interacting such that axial movement of the cutter results in rotation of the cutter.

In a further embodiment, the surgical device includes a handle and a trigger adjacent to the handle. The trigger is coupled to the cutter such that activation of the trigger causes the cutter or the stripping tube to move distally relative to the stripping tube or cutter, respectively. Further, the surgical device includes a lock assembly configured to selectively prevent activation of the trigger.

A method according to an exemplary aspect of the present disclosure includes, inter alia, separating a portion of a tendon by moving a stripping tube of a surgical device distally relative to the tendon, and severing the separated portion of the tendon by moving a cutter of the surgical device distally relative to the stripping tube.

In a further embodiment, the severing step includes pinching the separated portion of the tendon between a proximal edge of the stripping tube and a distal edge of the cutter.

In a further embodiment, the cutter rotates as the cutter moves relative to the stripping tube.

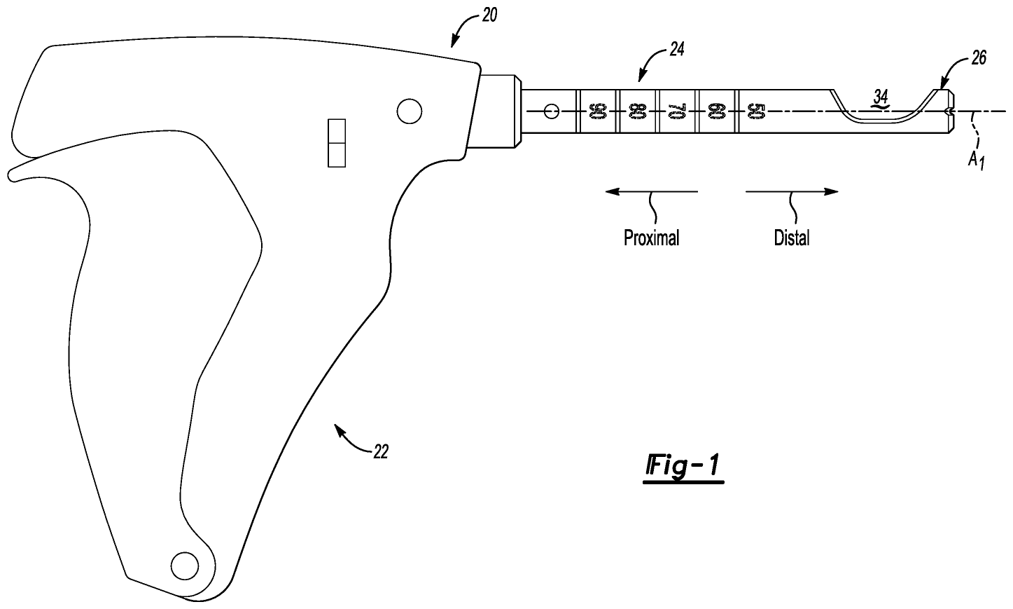

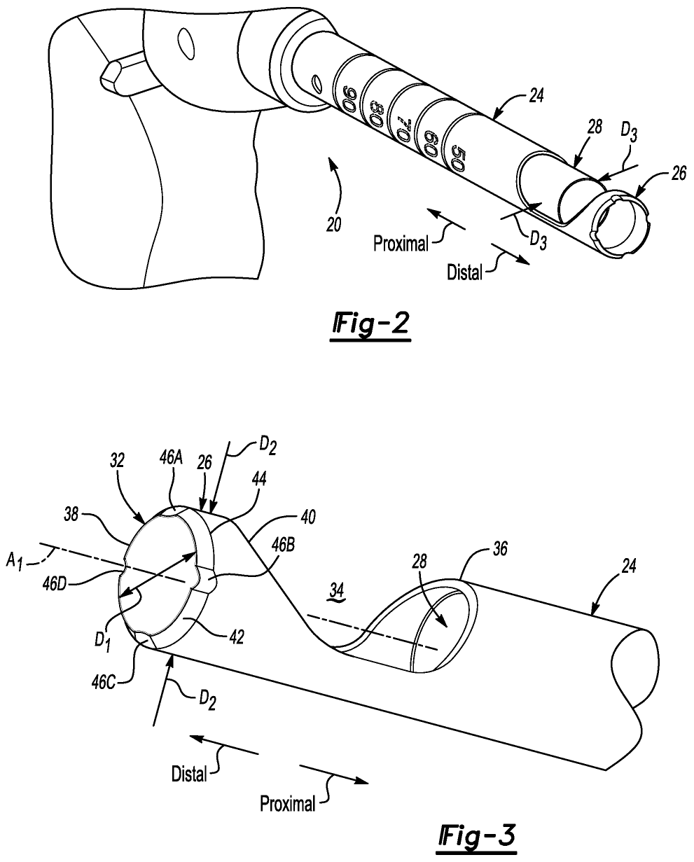

FIG. 1 illustrates an example surgical device 20. The surgical device 20 includes a handle 22 and a shaft 24 projecting distally from the handle 22. The “distal” and “proximal” directions are labeled across the various figures for purposes of explanation only. In this example, the shaft 24 includes a stripping tube 26 adjacent to a distal end thereof. The stripping tube 26 is configured to strip tendon. The surgical device 20 includes a cutter 28 (FIG. 2) moveable distally toward the stripping tube 26 to sever tendon. The detail of the surgical device 20, including the stripping tube 26 and cutter 28, will be described below.

With reference to FIG. 3, the shaft 24 comprises a substantially cylindrical body, which may be made of a metallic material. While the shaft 24 is substantially cylindrical in this example, the shaft 24 could exhibit different cross-sectional shapes, including being substantially square or rectangular. The shaft 24 extends along an axis A₁. Adjacent to a distal end 32 of the shaft 24, the shaft 24 includes a cutout 34 in a superior surface 36 (i.e., an upper surface) thereof. The cutout 34 allows a tendon to pass therethrough and defines the structure of the stripping tube 26, as will be discussed below.

The stripping tube 26 is a portion of the shaft 24 and extends completely around the axis A₁ in this example. The stripping tube 26 includes a distal edge 38 and a proximal edge 40. The distal edge 38 is the coextensive with the distal end 32 of the shaft 24, in this example. The proximal edge 40 of the stripping tube 26 is defined by a distal boundary of the cutout 34. An inner diameter D₁ of the stripping tube 26 intersects the axis A₁, such that the inner diameter D₁ of the stripping tube 26 is substantially circular in cross-section. In this way, the stripping tube 26 includes a window allowing a portion of a tendon to pass therethrough.

The distal edge 38 of the stripping tube 26 is configured to strip a portion of a tendon from adjacent tissue. In this example, the stripping tube 26 is tapered adjacent to the distal edge 38. In particular, an outer diameter D₂ of the stripping tube 26 gradually reduces in diameter throughout a tapered section 42. The tapered section 42 extends axially from the distal edge 38 to a location 44 proximal of the distal edge 38. The tapered section 42, in one example, is arranged such that the distal edge 38 is a sharp edge, meaning the distal edge 38 is tapered to a sharp point. In other examples, the distal edge 38 may be rounded or blunt while still capable of stripping a tendon. Further, in this example, tapered section 42 extends around the entirety of the axis A₁, but in other examples the tapered section 42 may extend only partially about the axis A₁.

In order to increase the ability of the stripping tube 26 to strip tendon, the stripping tube 26 may include one or more serrations adjacent to the distal edge 38. In this example, the stripping tube 26 includes four serrations 46A-46D. The serrations 46A-46D are equally spaced-apart from one another about the axis A₁. In this example, the serrations 46A-46D are notches extending proximally of the distal edge 38. This disclosure is not limited to any particular number or arrangement of serrations.

The substantially circular inner diameter D₁ allows one to harvest tendons that are substantially cylindrical, which allows one to harvest a properly-sized, cylindrical tendon without needing to approximate a cylindrical shape using a number of rectilinear cuts. Further, the inner diameter D₁ may be selected to correspond to a desired diameter of the harvested tendon. As examples, the inner diameter D₁ may be 7 mm, 10 mm, or 12 mm, although this disclosure is not limited to these particular dimensions. A surgeon may have a number of surgical devices 20 at their disposal, each of which has a stripping tube 26 of a different inner diameter D₁. The surgeon may select a surgical device 20 having an appropriately sized inner diameter D₁ for use in a particular procedure. In this way, the stripping tube 26 also acts as a sizing tube.

The cutter 28 is moveable distally toward the stripping tube 26 in order to sever, or cut, tendon. In this example, the cutter 28 comprises a substantially cylindrical body that is disposed about the axis A₁ and arranged within the shaft 24. The cutter 28 may be made of a metallic material. Like the shaft 24, the cutter 28 may also exhibit a different cross-sectional shape, such as being substantially square or rectangular in cross-section. This disclosure is not limited to a substantially cylindrical shaft 24 and cutter 28. The cutter 28 has an outer diameter D₃, which is substantially the same as the inner diameter D₁ of the stripping tube 26. In this example, the outer diameter D₃ is slightly less than the inner diameter D₁ to allow the cutter 28 to move relative to the shaft 24.

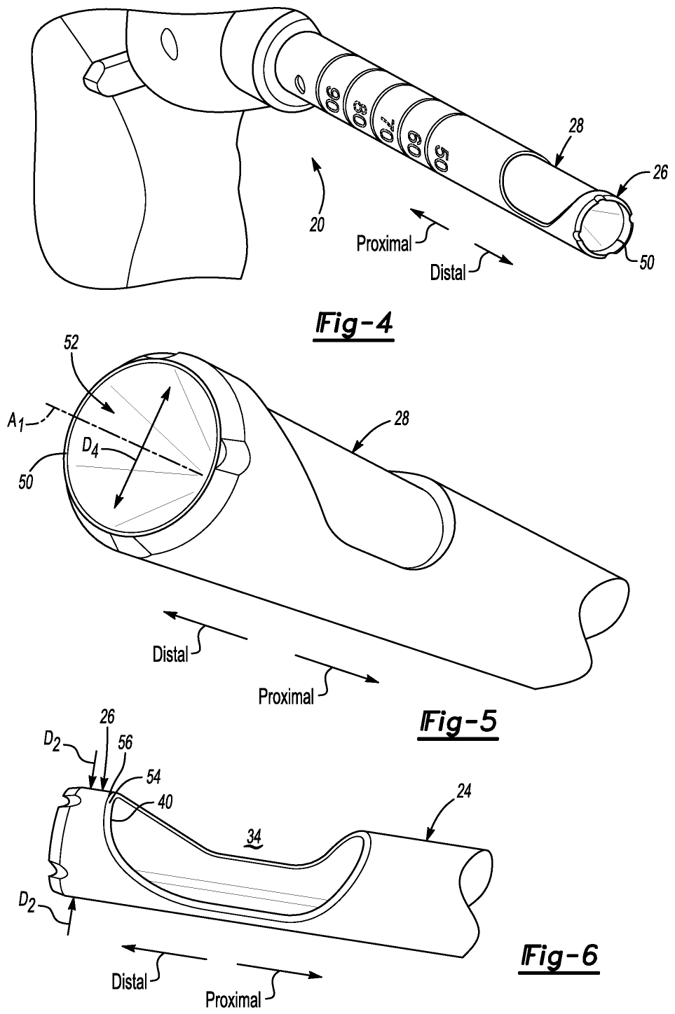

The cutter 28 is selectively moveable within the shaft 24 under the force of a trigger and one or more biasing elements, which will be discussed below. The cutter 28 is moveable from a neutral, resting position to a fully deployed position. The resting position is shown in FIGS. 1 and 3, wherein the entirety of the cutter is proximal of the cutout 34. FIGS. 4 and 5 illustrate the cutter 28 in the fully deployed position, in which the cutter 28 has moved distally relative to the resting position, and a distal edge 50 of the cutter 28 is distal of the cutout 34. For reference, FIG. 2 illustrates the cutter 28 in an intermediate position between the resting position and the fully deployed positions.

To increase the ability of the cutter 28 to sever a tendon, the cutter 28 is tapered adjacent to its distal edge 50. In this example, the outer diameter D₃ of the cutter 28 is substantially constant along the length of the cutter 28, and the cutter 28 includes a recess 52 (FIG. 5) adjacent to the distal edge 50. The recess 52 is tapered as it extends proximally from the distal edge 50. In particular, the recess 52 is defined by an inner diameter D₄, which gradually reduces from the distal edge 50 moving proximally. Thus, the recess 52 is substantially frustoconical in shape, although this disclosure is not limited to frustoconically-shaped recesses, and extends to other shapes such as rounded or squared recesses. The recess 52 may be such the distal edge 50 is a sharp edge, rounded edge, or blunt. In either instance, the recess 52 is such that the distal edge 50 effectively cuts tendon. While not shown in this embodiment, the cutter 28 may include one or more serrations, similar to the serrations 46A-46D, adjacent to the distal edge 50.

As a tendon is being stripped, the removed portion of the tendon exits the shaft 24 through the cutout 34. In order to sever the removed portion of the tendon, a user moves the cutter 28 distally toward the proximal edge 40 of the stripping tube 26. The tendon is severed by being pinched, and cut, between the proximal edge 40 of the stripping tube 26 and the distal edge 50 of the cutter 28. In one example, the proximal edge 40 of the stripping tube 26 is tapered to increase the ease of cutting tendon. As shown in FIG. 6, the stripping tube 26 may include a tapered section 54 beginning at a location 56 distal of the proximal edge 40. The outer diameter D₂ of the stripping tube 26 gradually reduces throughout the tapered section 54 moving proximally from location 56 to the proximal edge 40. The proximal edge 40 may be a sharp edge, rounded edge, or blunt. While in the illustrated embodiment both the proximal edge 40 and the distal edge 50 are tapered, in other examples only one of the proximal edge 40 and the distal edge 50 is tapered.

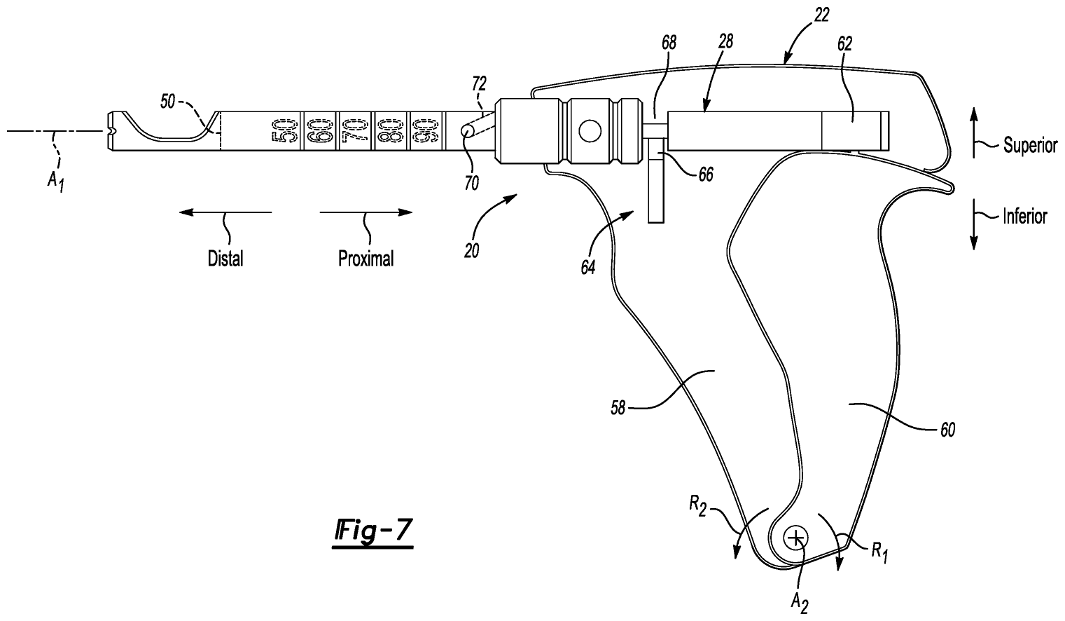

With reference to FIG. 7, an example arrangement of the handle 22 will now be described. In particular, an example arrangement configured to effect movement of the cutter 28 relative to the stripping tube 26 will be described. This disclosure is not limited to surgical devices including the particular arrangement of FIG. 7.

In FIG. 7, an exterior cover of the handle 22 is partially removed for ease of reference. The handle 22 includes a grip 58 and a trigger 60 rotatable relative to the grip about an axis A₂ adjacent to an inferior portion (i.e., bottom) of the grip 58. The “inferior” and “superior” directions are labeled in FIG. 7 for purposes of explanation only. The axis A₂ is normal to the axis A₁ in this example. The trigger 60 is proximal to the grip 58 and the handle 22 is arranged such that the trigger 60 rests between a user's thumb and index finger when a user grasps the grip 58.

The trigger 60 is mechanically coupled to the cutter 28 by way of a projection 62 projecting in the superior direction (i.e., an upper direction) from the remainder of the trigger 60. In this way, rotation of the trigger 60 about axis A₂ is translated into axial movement of the cutter 28 along the axis A₁.

In FIG. 7, the cutter 28 is in the resting position, in which the distal edge 50 of the cutter 28 is proximal of the cutout 34. One or more biasing elements may bias the cutter 28 and/or the trigger 60 toward the resting position. For example, the handle 22 may include one or more biasing elements urging the cutter 28 in the proximal direction, and the handle may include one or more biasing elements urging the trigger 60 to rotate in a first direction R₁ about the axis A₂, which in this example is a clockwise direction.

In order to move the cutter 28 distally toward the fully deployed position, a user applies a force to the trigger 60 to overcome the bias of the cutter 28 and/or the trigger 60 toward the resting position. To activate the trigger 60, a user applies a distal force onto the trigger 60, causing the trigger 60 to rotate in a second direction R₂ opposite the first direction R₁, which ultimately causes distal movement of the cutter 28. Deploying the cutter 28 is intuitive for the user because applying a distal force onto the trigger 60 is translated into distal movement of the cutter 28.

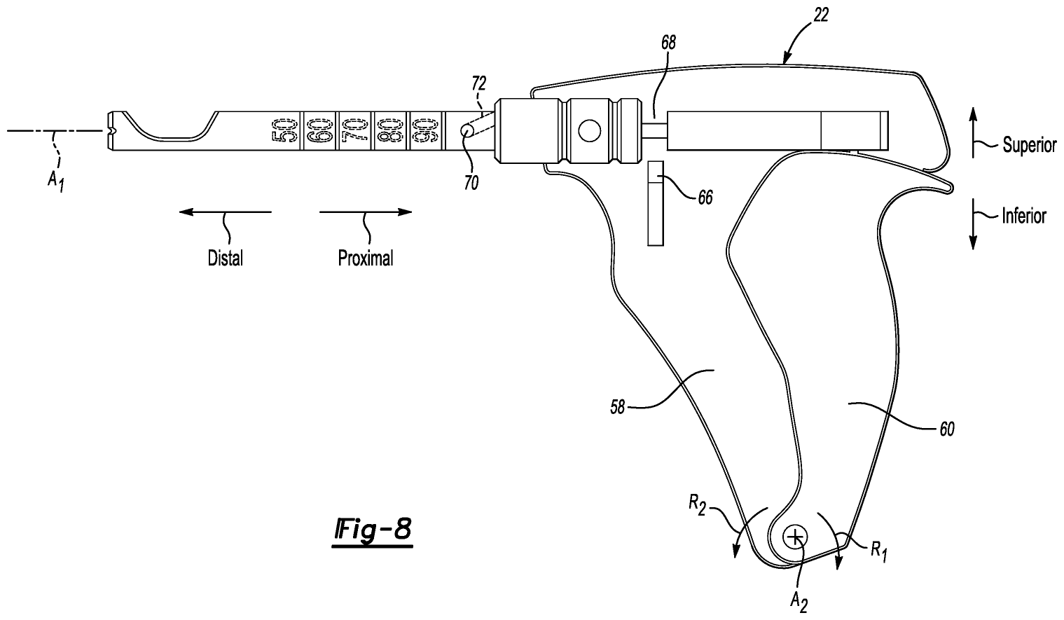

In an aspect of this disclosure, unintended deployment of the cutter 28 is prevented by way of a lock assembly 64. The lock assembly 64 includes a tab 66 moveable in the superior and inferior directions in and out of a slot 68 formed in the cutter 28. In FIG. 7, the lock assembly 64 is engaged, meaning the tab 66 is in the slot 68, which prevents axial movement of the cutter 28 and holds the cutter 28 in the resting position. In FIG. 8, the tab 66 has moved in the inferior direction relative to FIG. 7 to disengage the lock assembly 64, such as by a user applying a downward force to the tab 66. In FIG. 8, the tab 66 is not within the slot 68, and thus the tab 66 does not prevent axial movement of the cutter 28. This disclosure is not limited to surgical devices with lock assemblies.

With reference back to FIG. 7, in another aspect of this disclosure the surgical device 20 is arranged such that the cutter 28 rotates about the axis A₁ as it moves along the axis A₁. Rotating the cutter 28 relative to the stripping tube 26 may increase the ease of severing tendon. To rotate the cutter 28 in an embodiment, the surgical device 20 includes a pin 70 supported by the shaft 24 and projecting in a direction normal to the axis A₁. The cutter 28 includes at least one helical slot 72 formed therein. The helical slot 72 is shown in phantom in FIG. 7, and extends helically along the axis A₁. The pin 70 is arranged such that it projects into the helical slot 72. The interaction between the pin 70 and the helical slot 72 causes the cutter 28 to rotate about the axis A₁ as it moves axially along the axis A₁. The cutter 28 is not required to rotate in all examples, but, as mentioned, rotation may increase the ease of cutting. Further, it should be understood that this disclosure extends to configurations where the cutter 28 is fixed and the stripping tube 26 is configured to rotate about the axis A₁ as the stripping tube 26 moves axially relative to the cutter 28. While only one pin 70 and helical slot 72 are shown in FIG. 7, it should be understood that the shaft 24 and the cutter 28 may include additional pins and helical slots, respectively.

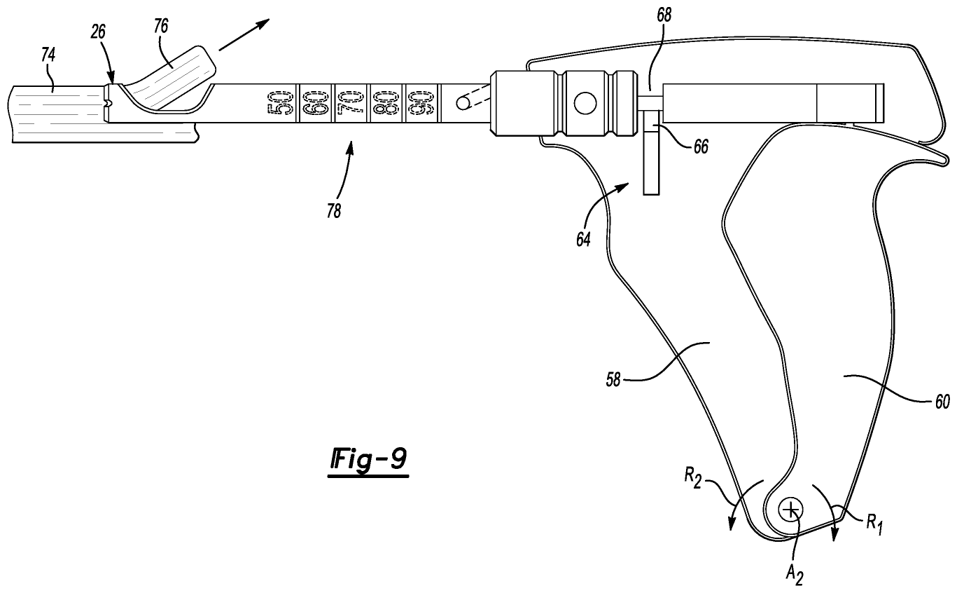

An example method of use will now be described with reference to FIGS. 9-11. In FIG. 9, the surgical device 20 is in contact with a tendon 74 and begins to strip the tendon. When stripping tendon 74 in this example, the cutter 28 is in the resting position, and the lock assembly 64 is engaged. Thereby, the tab 66 prevents unwanted movement of the cutter 28, which may otherwise sever a tendon prematurely.

In FIG. 9, a user strips the tendon 74 by bringing the distal edge 38 of the stripping tube 26 into contact with the tendon 74 and advancing the surgical device 20 distally along the tendon 74. A portion of the tendon 74 enters the stripping tube 26 and becomes partially separated from the remainder of the tendon 74. The partially separated portion of the tendon is labeled with reference numeral 76. The portion 76 is substantially cylindrical in cross-section by virtue of the substantially circular cross-section of the stripping tube 26.

The tendon 74 may be a quadriceps tendon in one example. This disclosure is not limited to any particular type of tendon, however. Further, this disclosure may be used with other types of soft tissue and is not limited to use with tendons.

In the example method, a user continues advancing the surgical device 20 distally until the portion 76 is of a desired graft length. The user may measure the portion 76 using markings 78 on the exterior of the shaft 24. The markings 78 correspond to a distance proximal of the cutout 34, and may be in millimeters spaced-apart by units of 10 (e.g., 50, 60, 70, 80, 90). The markings 78 are shown in phantom in the figures to avoid confusion with the other reference numerals used in the figures and discussed herein. The shaft 24 need not include markings in all examples.

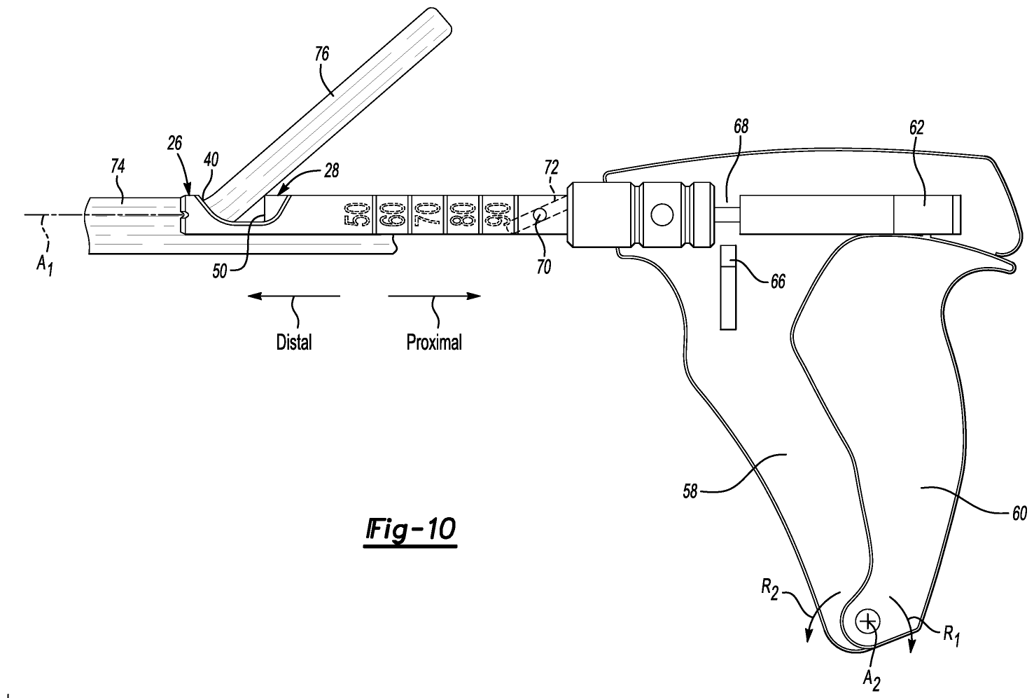

In FIG. 10, the user has advanced the surgical device 20 distally to a point where the portion 76 of the tendon 74 will provide an adequate graft length. Thus, the user has disengaged the lock assembly 64 by moving the tab 66 in the inferior direction and has begun advancing the cutter 28 distally. The user has activated the trigger 60 by squeezing the grip 58 and trigger 60 together, thereby rotating the trigger 60 in the direction R₂ about the axis A₂. Such rotation results in distal movement of the cutter 28 along the axis A₁ by virtue of projection 62 being mechanically coupled to the cutter 28. The axial movement of the cutter 28 also results in rotational movement of the cutter 28 about the axis A₁ by virtue of the arrangement of the pin 70 and the helical slot 72.

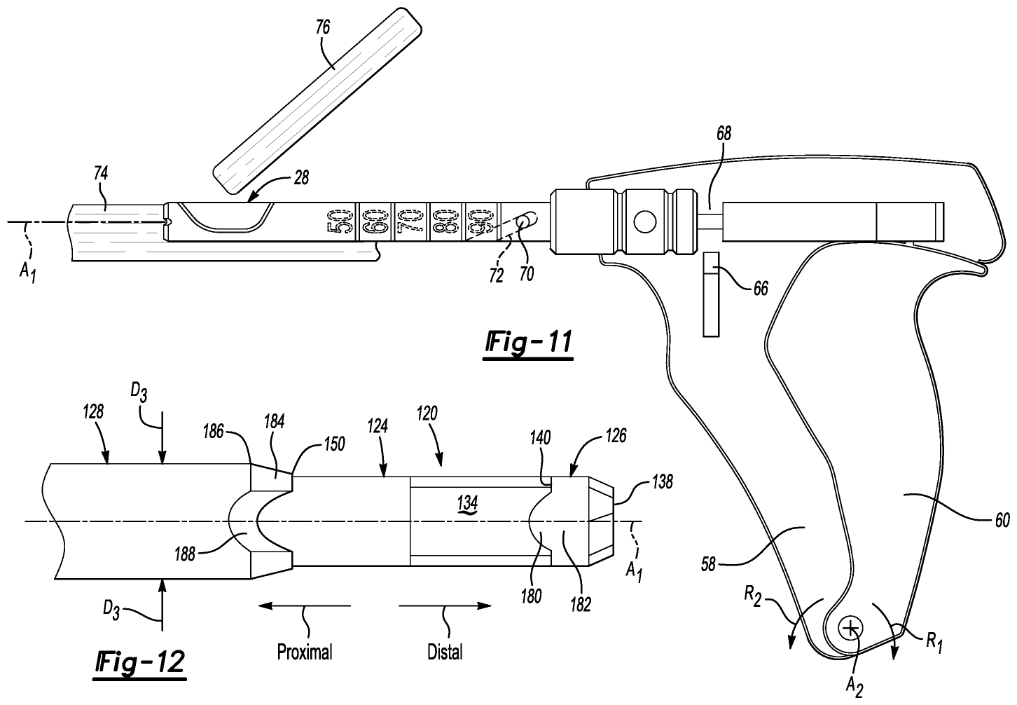

Continued squeezing causes additional distal movement of the cutter 28, which in turn causes the portion 76 of the tendon 74 to become pinched between the distal edge 50 of the cutter 28 and the proximal edge 40 of the stripping tube 26. Ultimately, the portion 76 is completely severed from the remainder of the tendon 74, as shown in FIG. 11, in which the cutter 28 is in the fully deployed position. Among other benefits, the surgical device 20 is intuitive and allows one to harvest tendon using fewer instruments than prior techniques.

FIG. 12 is a top view of a portion of another example surgical device 120. To the extent not otherwise described or shown, the surgical device 120 corresponds to the surgical device 20, with like parts preappended with a “1.”

Unlike the surgical device 20, in which the cutter 28 is disposed circumferentially within the shaft 24, the surgical device 120 is arranged such that the cutter 128 is disposed circumferentially outward of the shaft 124. The cutter 128 may be moveable between a resting position and a fully deployed position in substantially the same way as described relative to FIGS. 7-11. The cutter 128 may rotate as it moves axially, as in the previous embodiment. Alternatively, the cutter 128 need not rotate.

In an additional embodiment, the stripping tube 126 is substantially similar to that of the stripping tube 26, with the exception of the proximal edge 140. In FIG. 12, the proximal edge 140 of the stripping tube 126 is not tapered. Rather, the proximal edge 140 is blunt and includes a rounded projection 180 projecting proximally of a superior surface 182 of the stripping tube 126. In a variation of this embodiment, the proximal edge 140 may be tapered, in generally the same way as the proximal edge 40. In still another variation, the proximal edge 140 does not include the rounded projection 180.

The distal edge 150 of the cutter 128 is also arranged differently than in the previous embodiment. In FIG. 12, the outer diameter D₃ of the cutter 128 includes a tapered section 184 extending from the distal edge 150 to a location 186 proximal of the distal edge 150. The outer diameter D₃ gradually reduces in thickness moving distally throughout the tapered section 184. The cutter 128 also includes at least one serration 188 adjacent to the distal edge 150. In FIG. 12, the cutter 128 includes only one serration 188, which is a notch formed in the distal edge 150 in this example. The serration 188 is symmetrical about a plane passing through the axis A₁ and bisecting the surgical device 120. The serration 188 is aligned with the projection 180 such that a common plane bisects both the projection 180 and the serration 188. The projection 180 and the serration 188 cooperate with one another to provide effective cutting of a tendon. While only one serration 188 is shown in FIG. 12, the cutter 128 may include additional serrations. Alternatively, the cutter 128 may not include any serrations.

It should be understood that terms such as “distal,” “proximal,” “superior,” “inferior,” etc., have been used herein for purposes of explanation, and should not be considered otherwise limiting. Terms such as “generally,” “substantially,” “about,” “slightly,” etc., are not intended to be boundary less terms, and should be interpreted consistent with the way one skilled in the art would interpret those terms.

Although the different examples have the specific components shown in the illustrations, embodiments of this disclosure are not limited to those particular combinations. It is possible to use some of the components or features from one of the examples in combination with features or components from another one of the examples. In addition, the various figures accompanying this disclosure are not necessarily to scale, and some features may be exaggerated or minimized to show certain details of a particular component or arrangement.

One of ordinary skill in this art would understand that the above-described embodiments are exemplary and non-limiting. That is, modifications of this disclosure would come within the scope of the claims. Accordingly, the following claims should be studied to determine their true scope and content.

1. A surgical device, comprising:
a stripping tube configured to strip a tendon; and
a cutter moveable distally toward the distal end of the stripping tube to sever a portion of the tendon.

2. The surgical device as recited in claim 1, wherein the stripping tube is tapered.

3. The surgical device as recited in claim 2, wherein the distal edge of the stripping tube comprises a plurality of serrations.

4. The surgical device as recited in claim 1, wherein the stripping tube includes a window which is substantially circular in cross-section.

5. The surgical device as recited in claim 1, wherein the stripping tube comprises a window allowing the portion of the tendon to pass therethrough.

6. The surgical device as recited in claim 5, wherein the cutter is disposed circumferentially.

7. The surgical device as recited in claim 6, wherein the distal end of the cutter includes a frustoconical recess.

8. The surgical device as recited in claim 6, wherein the distal end of the cutter is tapered.

9. The surgical device as recited in claim 1, wherein the cutter is moveable such that a tendon may be severed between a proximal edge of the stripping tube and a distal edge of the cutter.

10. The surgical device as recited in claim 9, wherein the proximal edge of the stripping tube is tapered and the distal edge of the cutter is tapered.

11. The surgical device as recited in claim 10, wherein the distal edge of the cutter includes at least one serration.

12. The surgical device as recited in claim 11, wherein the at least one serration is a single serration substantially symmetrical about a centerline of the surgical device.

13. The surgical device as recited in claim 1, wherein the stripping tube and cutter are disposed about a common axis.

14. The surgical device as recited in claim 13, wherein the cutter is configured to rotate about the common axis as the cutter moves axially relative stripping tube.

15. The surgical device as recited in claim 14, wherein the stripping tube is configured to rotate about the common axis as the stripping tube moves axially relative to the cutter.

16. The surgical device as recited in claim 14, wherein the cutter includes a helical slot receiving a pin, the pin and helical slot interacting such that axial movement of the cutter results in rotation of the cutter.

17. The surgical device as recited in claim 1, further comprising:
a handle and a trigger adjacent to the handle, the trigger coupled to the cutter such that activation of the trigger causes the cutter or the stripping tube to move distally relative to the stripping tube or cutter, respectively; and
a lock assembly configured to selectively prevent activation of the trigger.

18. A method, comprising:
separating a portion of a tendon by moving a stripping tube of a surgical device distally relative to the tendon; and
severing the separated portion of the tendon by moving a cutter of the surgical device distally relative to the stripping tube.

19. The method as recited in claim 18, wherein the severing step includes pinching the separated portion of the tendon between a proximal edge of the stripping tube and a distal edge of the cutter.

20. The method as recited in claim 18, wherein the cutter rotates as the cutter moves relative to the stripping tube.
